(19) United States
(12) Patent Application Publication
(10) Pub. No.: US 2025/0263804 A1
(43) Pub. Date: Aug. 21, 2025

(54) METHOD FOR DETECTING A TARGET GENOME

(71) Applicants: UNIVERSITÀ DEGLI STUDI DI MESSINA, MESSINA (IT); ALMA MATER STUDIORUM - UNIVERSITÀ DI BOLOGNA, BOLOGNA (IT)

(72) Inventors: SABRINA CONOCI, CATANIA (IT); ENRICO RAMPAZZO, BOLOGNA (IT); GIOVANNI VALENTI, BOLOGNA (IT); FRANCESCO PAOLUCCI, NOVENTA PADOVANA (IT); ALESSANDRA ZANUT, PORDENONE (IT); LUCA PRODI, BOLOGNA (IT); SALVATORE PETRALIA, MESSINA (IT)

(21) Appl. No.: 18/579,720

(22) PCT Filed: Jul. 4, 2022
(86) PCT No.: PCT/IT2022/050195

§ 371 Date: Jan. 16, 2024

(30) Foreign Application Priority Data
Jul. 13, 2021 (IT) 102021000018422

(51) Int. Cl. C12Q 1/70; C12Q 1/6825

(52) U.S. Cl. CPC C12Q 1/706; C12Q 1/6825

(57) ABSTRACT

The present invention relates to a method allowing a rapid and sensitive genome molecular detection, by the detection of nucleic acids thereof. Specifically, the method combines the cooperative hybridization process of a macromolecular genetic target on the electrode surface derivatized with an electrochemiluminescence (ECL)-based ultrasensitive detection. The method allows to directly detect a target genome without any amplification. Therefore, the method of the invention can be considered as an amplification-free approach, in particular a PCR-free approach.

The present invention relates to a method allowing a rapid and sensitive genome molecular detection, by the detection of nucleic acids thereof. Specifically, the method combines the cooperative hybridization process of a macromolecular genetic target on the electrode surface derivatized with specific probes and combined with electrochemiluminescence (ECL)-based ultrasensitive detection. The method allows to directly detect a target genome without any analyte amplification. Therefore, the method of the invention can be considered as an amplification-free approach, in particular a PCR-free approach.

The molecular analysis of nucleic acids (DNA and RNA) is nowadays crucial in many medical fields for early and accurate diagnosis, personalized therapy, and preventive screening. This is particularly relevant in the field of the infectious diseases, as it has become clear after the pandemic infection of COVID-19 that affected worldwide more than 22 millions of people with more than 800 thousands of deaths. This urgency is particularly evident, in general, for the states where these diseases catastrophically affect the health of population, and in particular for the developing countries where infectious diseases are the cause of mortality and morbidity for more than half of all infant deaths.

In this context, molecular analysis provides relevant clinical advantages with respect to the traditional laboratory methods (bacterial cultures or antibody detection) being much faster (hours versus days for bacterial cultures), specific (allowing the detection of genotypes), sensitive (achieves the detection of few copies of pathogens in a sample) and accurate (able to detect not only the “presence” of the microorganism (DNA) but also its vitality (mRNA)). However, molecular methods need amplification through the well-known Polymerase Chain Reaction (PCR) that is intrinsically quite laborious (involving several analytical laboratory steps) and expensive (about $20-80 per sample) and, as a consequence, currently exclusively performed in specialized centralized laboratories. Therefore, although the current molecular PCR-based methods are well-established and consolidated methodologies, they are not suitable to be used by unskilled personnel near the patient at competitive costs (few dollars per test). This represents a strong limitation for its massive use limiting, de facto, the potential of molecular analysis for the human health. As proof of this, there is the current difficult of a massive and real time diagnosis of COVID for prompt infection management and prevention (the molecular analysis takes at least 1 day). Therefore, the development of new molecular methods allowing rapid and sensitive pathogens detection represent a significant breakthrough in the molecular diagnostic field.

Therefore, there is the need of an amplification-free approach, in particular a PCR-free approach, however that is very challenging, in particular in the case of infectious diseases where the target concentration could be as low as few copies of bacteria or viruses, which allows to fulfil the sensitive requirements of LoD (10 copies of target/reactions) and without requiring any sample pre-treatment of labelling.

The technical problem addressed by the present invention is to provide a detection method based on an amplification-free approach, in particular PCR-free approach, allowing the direct, rapid and sensitive pathogen molecular detection.

Such problem is solved through the method of the present invention, the method being defined by the attached claims, whose definitions are integral part of the description.

The Applicant has now found a new molecular method allowing a rapid and sensitive whole genome molecular detection, by the detection of nucleic acids thereof. Specifically, the method combines the cooperative hybridization process of the whole macromolecular genetic target on the electrode surface derivatized with specific probes and combined with an electrochemiluminescence, ECL-based ultrasensitive detection. The method allows to directly detect a target whole genome of an organism, in particular a pathogen genome, without any analyte amplification. Therefore, the method of the invention can be considered as an amplification-free approach, in particular a PCR-free approach. Specifically, it was based on the combination of Surface Cooperative Hybridization (SCH) of a target whole genome by complementary ss-oligo probes, in particular DNA, RNA, or PNA, anchored on an ECL electrode surface. In fact, these probes are designed to recognize specific sequence portion of the target whole genome. In this way, they are capable to guarantee the simultaneous hybridization with the target genome (a sequence gap between the two probes can be maintained). Upon hybridization, the entire genome of the target is recognized by the surface probes that independently hybridize the two complementary filaments of the genome anchoring it at the electrode surface forming a supramolecular complex.

Therefore, according to a first aspect, the present invention relates to a method according to claim 1.

Advantageously, the method of the invention is very high-sensitive capable of fulfilling the requirements of LoD (10 copies of target/reactions). The method of the invention can also be easily integrated in portable and low-cost devices thus enabling the massive use for diagnostic screening. Therefore, it allows the creation of cheap Point of Care testing (PoCT). Furthermore, the method of the invention does not require any pre-treatment and labelling.

Further features and advantages of the method according to the invention will be evident from the description of the embodiment of the invention, which is to be intended as illustrative, but not limiting, purpose.

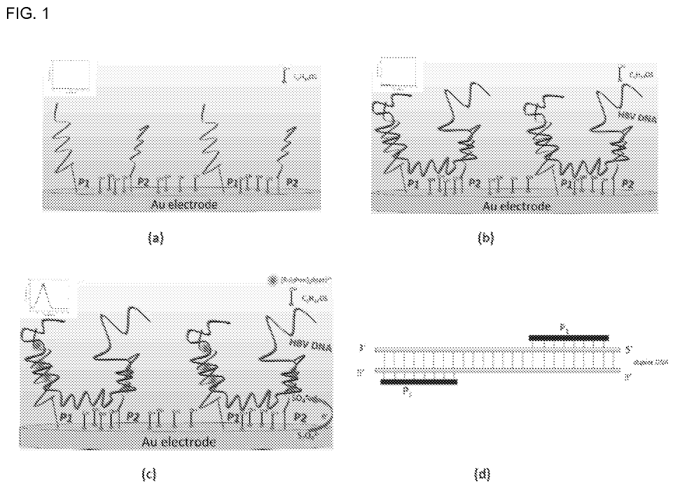

FIG. 1: Experimental procedure for the creation of molecular recognition based on supramolecular complex formation. (a) Immobilization of specific probes and thiol in the surface of gold electrode (b) Recognition of ds-DNA HBV and the supramolecular complex formation (c) Intercalation of [Ru(phen)₂dppz]²⁺ and the HBV quantification (d) P1 and P2 probes design for cooperative hybridization.

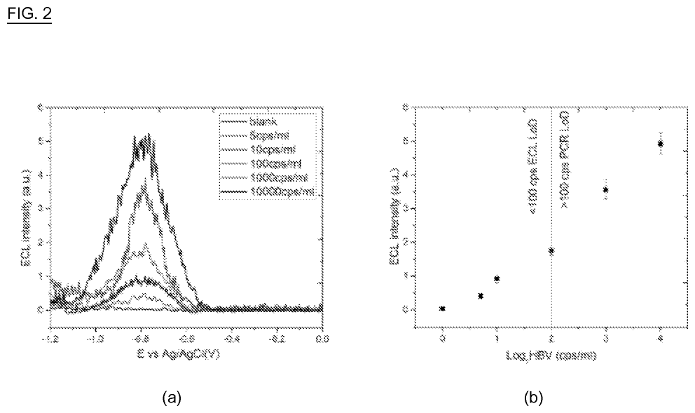

FIG. 2: ECL intensity vs potential for different concentration of synthetic genome HBV; (a) 0-10000 cps/ml (b) ECL intensity vs Log HBV concentration in PBS 0.1 M and 50 mM K₂S₂O₈. Working Au electrode with P1 and P2 formation vs Ag/AgCl reference electrode with scan rate 0.3 V/s. PMT bias of 750 V.

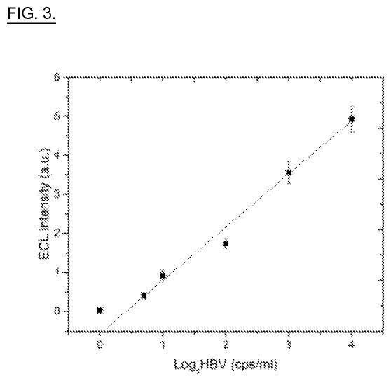

FIG. 3: Calibration plot analysis and dependence of the response of the electrode on concentration of HBV genome with standard deviation of ±0.075-0.321. In PBS 0.1 M, 14 μM intercalated [Ru(phen)₂dppz]²⁺ and 50 mM K₂S₂O₈. Working Au electrode with P1 and P2 formation vs Ag/AgCl reference electrode with scan rate 0.3 V/s. PMT bias of 750 V.

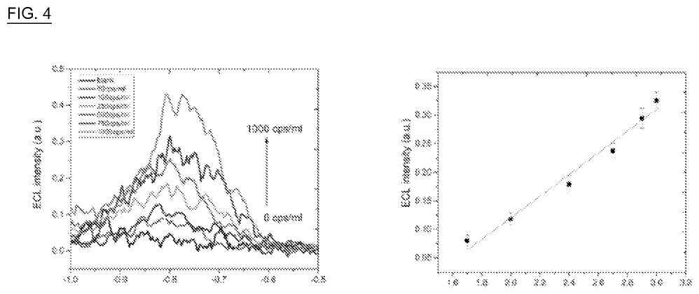

FIG. 4: ECL intensity vs potential for different concentration of extracted HBV genome; (a) 0-10000 cps/ml (b) calibration curve of Log (HBV concentration, expressed in cps/ml) in PBS 0.1 M, 14 μM intercalated [Ru(phen)₂dppz]²⁺ and 50 mM K₂S₂O₈. Working Au electrode with P1 and P2 formation vs Ag/AgCl reference electrode with scan rate 0.3 V/s. PMT bias of 750 V.

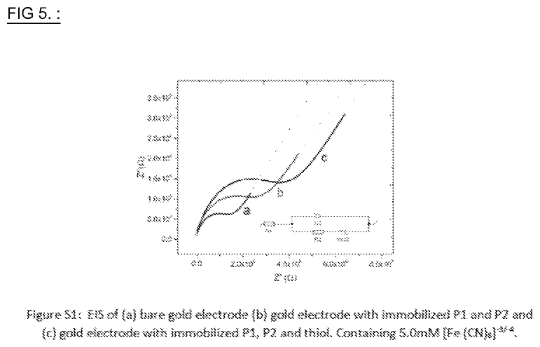

FIG. 5: reports the figure indicated in the example part as FIG. S1 relating to EIS of three electrodes-type.

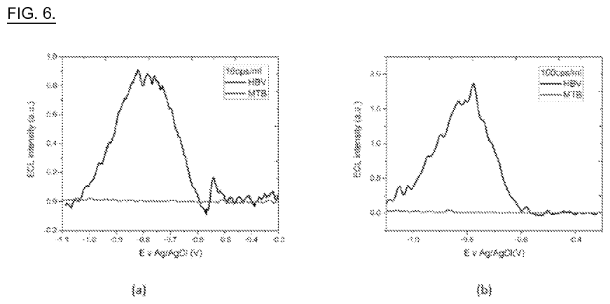

FIG. 6: reports the figure indicated in the example part as FIG. S2 and FIG. S3.

For the scopes of the invention, the following definitions used in the present description and attached claims are provided.

The method of the invention is based on electrochemiluminescence, ECL, which is the analytical technique based on a luminescent phenomenon induced by electrochemical stimulus. In particular, with the ECL co-reactant mechanism the excited state of a luminophore is generated through the reaction between the radical of luminophores and reactive intermediates of the co-reactant produced by the electrochemical oxidation or reduction. In particular, the method of the invention is based on the molecular recognition of genome molecules by the formation with specific probes anchored at electrode surface and detection via ECL luminophore. The ECL signal increases in the presence of target if intercalation is promoted by its presence.

An object of the present invention is a method based on ECL for detecting an analyte in a sample, wherein the analyte is a target whole genome of an organism, comprising the steps of:
a) contacting the sample containing the whole genome with at least two single-strand nucleic acid probes, wherein each of the at least two single-strand nucleic acid probes is complementary to a corresponding portion of said target whole genome;
b) adding an ECL active luminophore, wherein said ECL active luminophore is a ECL intercalative luminophore;
c) determining a luminescent signal generated by the ECL active luminophore;
wherein said target whole genome is selected from a DNA or RNA genome.

According to a preferred aspect, the target whole genome can be single stranded (ss-) or double stranded (ds-).

According to a preferred aspect, the single-strand nucleic acid probes are DNA, RNA or PNA.

According to a preferred aspect, the single-strand nucleic acid probes are 6-18 residue probes.

According to a preferred aspect, the single-strand nucleic acid probes are immobilized by a surface linker.

According to a preferred aspect, when the target whole genome is a double-stranded genome, at least one of the at least two single-strand nucleic acid probes is complementary to a portion of the parallel strand of the double-stranded target whole genome and at least one of the at least two single-strand nucleic acid probes is complementary to a portion of the anti-parallel strand of the double-stranded target whole genome. Preferably, the double-stranded target whole genome is ds-DNA.

According to a preferred aspect, with reference to the single-strand nucleic acid probes, which can be also indicated as ss-nucleic acid probes, they are preferably single-strand nucleic acid oligo probes (ss-nucleic acid oligo probes).

As said above, the method of the present invention allows to detect a target whole genome of an organism. Preferably, said organism is a microorganism, even more preferably a virus or a bacterium.

According to a preferred aspect, the target whole genome is a viral or bacterial genome. Preferably, the target whole genome is a viral or bacterial, parasite, eukaryotic cell whole genome.

Generally, the number of single-strand nucleic acid probes can be suitably adjusted based on the type of the target whole genome.

Furthermore, the selectivity of the method of the invention depends on the single-strand nucleic acid probes which can be selectively choses depending on the whole target genome.

As intended herein, the term target whole genome of an organism (also indicated herein simply as target whole genome) has the same meaning of target full genome, target complete genome, or target entire genome of an organism.

In a preferred aspect, the target double-strand nucleic acid is viral nucleic acid, more preferably the viral nucleic acid is of Hepatitis B Virus (HBV). When the target whole genome is of HBV, the at least two single-strand nucleic acid probes are: at least one sequence is the sequence SEQ. ID. NO. 1 (GGTGAGTGATTGGAGGTT) and at least one sequence is SEQ. ID. NO. 2 (CACATCAGGATTCCTAGG). Preferably a linker can be used, more preferably the linker is HS—(CH₂)₆—. According to a preferred aspect, a linker is used with said sequence SEQ. ID. NO. 1 (GGTGAGTGATTGGAGGTT) and SEQ. ID. NO. 2 (CACATCAGGATTCCTAGG), preferably said linker being HS—(CH₂)₆—.

According to a preferred aspect, the single-strand nucleic acid probes of step a) are two. Preferably said two single-strand nucleic acid probes have sequences: SEQ. ID. NO. 1 (GGTGAGTGATTGGAGGTT) and SEQ. ID. NO. 2 (CACATCAGGATTCCTAGG).

According to a preferred aspect the electrode is a gold electrode. Preferably such electrode is miniaturized.

The ECL-based method of the invention allows the diagnosis and quantification based on the combination between surface whole target genome immobilization and ECL transduction by ECL-intercalative luminophore that intercalates on the grooves of DNA.

Therefore, the ECL-intercalative luminophore is an intercalating agent, which exhibits extraordinary environmental sensitivity of its luminescence lifetime and quantum yield. The ECL active molecule (alternatively indicated as ECL active luminophore or ECL active), is preferably a Ru(II), Ir(III), Re(I), or Os(II) complex, more preferably Ru(II) complex. Preferably, the ECL active luminophore is a Ru(II), Ir(III), Re(I), or Os(II) coordination complex.

According to a preferred aspect, the ECL active luminophore has a dppz ligand. Preferably, the ECL active luminophore is a Ru(II) complex with dppz ligand (dppz=dipyrido[3,2-a:2′,3′-c]phenazine), preferably Ru(bpy)₂dppz]²⁺ or [Ru(phen)₂dppz]²⁺ (phen=1,10-Phenanthroline, bpy=2,2′-bipyridine). Other preferred ligands are 1,10-phenanthroline; quinoxalino[2,3-f][1,10]phenanthrolin. Preferably, the ECL active luminophore is a Ru(II)- or Os(II)-complex.

According to a preferred aspect the ECL co-reactant is a sacrificial reagent that after oxidation or reduction is able to generate reactive radicals for the generation of ECL signal. The ECL co-reactant is preferably K₂S₂O₈. Other preferred co-reactant are tertiary amines, oxalate (e.g. 2-(dibutylamino)ethanol)oxalate)) and hydrogen peroxide.

### EXAMPLES

### Chemicals and Materials

All the reagents used was of analytical grade. Phosphate buffer Solution (PBS) of different concentrations (0.1M and 0.01M) and different pHs (5.5, 6.5, 7.4 and 8.5), potassium persulfate (K₂S₂O₈) >99%, sulfuric acid (H₂SO₄) 98%, Potassium hexacyanoferrate (III) (K₃[Fe(CN)₆]^{-3/-4}) and Ethanol (C₂H₆O) 100% were purchased from Sigma-Aldrich, Fetal Bovine Serum (FBS) produced by Gibco∃ 100% was purchased from ThermoFisher Scientific. [Ru(phen)₂dppz]²⁺ was synthetize as previously described, 6-Mercapto-1-hexanol (C₆H₁₄OS) >97% was purchased from Fluka. Hepatitis B virus (HBV) clone complete genome (HBV clone analytical sample) was purchased from Clonit (ref 05960467) and it is the HBV genome (3.2 kbps) inserted into plasmid PBR322 vector (3.8 kbps). It is provided in a TE (Tris 10 mM, EDTA 1 mM, pH=8) solution. Micobacterium Tuberculosis (MTB) clone complete genome was obtained from Clonit (ref 05960564) provided in aTE (Tris 10 mM, EDTA 1 mM, pH=8) solution to test the selectivity of the system. Complementary P1 probe (HS—(CH2)6-GGTGAGTGATTGGAGGTT) and P2 Probe (HS—(CH₂)6-CACATCAGGATTCCTAGG) were purchased from MWG (Germany). HBV genome extraction from human blood (extracted real HBV genome sample) was carried out using Qiagen QIAamp DNA Mini Kit (ref. 51306), following the Instructions for Use.

### Apparatus and Measurements

All ECL and electrochemical measurements were conducted with a potentiostat (PGSTAT302N, Metrohm) using a single-compartment three-electrode Teflon cell with Au electrode (CH Instrument 101) as the working electrode (WE), a Pt wire as the counter electrode (CE), and an Ag/AgCl (saturated KCl) electrode as the reference electrode (RE). The ECL signal was measured with a photomultiplier tube (PMT, Hamamatsu R928) placed at a fixed height from the electrochemical cell, inside a dark box. A high-voltage power supply socket assembly with a transimpedance amplifier (Hamamatsu C6271) was used to supply 750 V to the PMT, using an external trigger connection to the potentiostat DAC module. Light/current/voltage curves were recorded by collecting the amplified PMT output signal with the ADC module of the potentiostat. ECL spectra were collected by a SEC2000 Spectra system UV-visible spectrophotometer (ALS Co. Ltd., Japan). However, as electrochemical technique it was used the cyclic voltammetry applying negative potential in our system, with scan rate 0.3 V/s. For the Electrochemical Impedance Spectroscopy (EIS) was used buffer solution of PBS 0.1M, pH 5.5 containing 5.0 mM (K₃[Fe(CN)₆]^{-3/-4}). All EIS measurements have been done in the range of frequency 0.1 MHz-1 Hz, thus the current range has been kept equivalent at 1 mA. Finally, the value of open circuit potential (Eocp) had the same value of 0.228V throughout the measurement.

### Fabrication of HBV DNA Sensor

The gold electrode was polished with 0.3, and 0.05 μm alumina slurry in order to obtain a mirror surface and then washed by sonication in a 50:50 ethanol and deionized water for 5 min. Furthermore, the gold electrode was cleaned electrochemically to cyclic potential scanning between −1 and 1 V in 0.5M H₂SO₄ solution for 10 cycles. Finally, the gold electrode was dried with Ar gas. Next, a buffer solution of PBS 0.01 M at pH 7.4 was prepared, in which the P1 and P2 thiol 5′-terminated probes were dissolved at a concentration of 10 μM and immobilized in the surface according to Au—S chemistry. In this solution the gold electrode was immersed and kept for 4 hours at room temperature (RT) conditions. However, the surface of gold electrode had been washed with PBS 0.01M at pH 7.4 using few drops. In the sequel an overnight blocking procedure took place using a solution of PBS 0.01 M at pH 7.4 with 10 μM of C₆H₁₄OS was prepared, such as to fill the gaps between P1 and P2 specifics probes (FIG. 1a). In this way, it was easier to modify the Au electrode/P1P2 surface making the supramolecular complex formation adding ds-DNA of HBV. After blocking modification, different concentration of ds-DNA HBV (1-10000 cps/ml) were prepared at PBS 0.01M at pH 5.5, in this way every electrode is immersed into these solutions at 50° C. in the chamber. The achievement of the supramolecular complex formation has been achieved after 3 hours of this step (FIG. 1b). However, a stock solution of 14 μM [Ru(phen)₂dppz]²⁺ it was prepared at PBS 0.01M at pH 5.5 for the intercalation of [Ru(phen)₂dppz]²⁺ in the grooves of ds-DNA. The electrodes were remained immersed in the [Ru(phen)₂dppz]²⁺ for 2 hours until the intercalation was completed. After the described procedure, the HBV DNA sensor was ready to be used (FIG. 1c).

### Analytical Test Procedure

The gold electrode derivatized with P1 and P2 probes was cleaned using PBS 0.01M pH 7.4 and the was immersed in the solution of known concentration of HBV clone, in the chamber at 50° C. for 4 hours. Then, the supramolecular complex formation had been achieved and the gold electrode was cleaned with PBS 0.01M pH 5.5. The acid value of pH protonates the -G and -C groups of ds-DNA (promoting the supramolecular complex triplex-DNA formation) to have a stronger bonding between the P1 and P2 probes and ds-DNA of HBV. Finally, a solution of PBS 0.01M pH 5.5 containing [Ru(phen)₂dppz]²⁺14 M was prepared and the gold electrode was immersed for 2 hours. After the intercalation of [Ru(phen)₂dppz]²⁺ in the grooves of hybridized DNA, the Au electrode was ready for the ECL procedure. Analytically, HBV genome samples (1, 5, 10, 100, 1000, 10000 copies/reaction) were prepared by diluting the starting clone solution (106 copies) in deionized water. However, for the real sample analysis, was used HBV genome synthetic clone extracted from human blood (50-100-250-500-750-1000 copies/reaction) were prepared by diluting the starting clone solution (2000 copies) in deionized water. Finally, for the ECL experiments using biological fluids such as FBS and using real samples, we follow the same experimental procedure as it is described above. The different part is the use of FBS instead of PBS for the analysis in biological fluids and the different concentrations of extracted HBV genome.

### Comments of the Experimental Results

The method of the invention allows to directly detect a target genome, in particular a pathogen genome, more in particular HBV genome, without any amplification. With particular reference to HBV detection, specifically the method was based on the combination of Surface Cooperative Hybridization (SCH) of the double stranded HBV genome (ds-DNA genome) by two complementary ss-oligo probes anchored on an ECL electrode surface. The two oligo probes (P1 and P2) were designed to be complementary with a portion of a single filament of the ds target genome. These probes are designed to recognize specific sequences on both parallel and anti-parallel strands of the same target double strand genome. In this way, they are capable to guarantee the simultaneous hybridization with the two strands of the genome target, a sequence gap of 138 bps between the two probes was maintained as it is presented in figure id. This sequence gap is important to minimise the steric hindrance during the molecular recognition process between probes and genome. Upon hybridization, the entire genome of the target is recognized by the surface probes that independently hybridize the two complementary filaments of the genome anchoring it at the electrode surface forming a supramolecular complex-DNA target-probe structure. The detection of HBV genome from both the analytical sample and real sample extracted from human blood. Also, as a ECL active complex (luminophore) was used a ruthenium compound ([Ru(phen)₂dppz]²⁺) which is a specific ECL intercalative molecule such as Ruthenium (II) complexes with dppz ligand exhibit extraordinary environmental sensitivity of their luminescence lifetimes and quantum yields.

### Characterization of Modified Electrode

The first part of this work was focused on the optimization of the chemical treatment of the surface of gold electrode for the effective anchoring of P1 and P2 probes. The surface of the electrode which was used for the ECL biosensing was characterized by Electrochemical Impedance Spectroscopy (EIS). FIG. S1, demonstrates the Nyquist plots of the EIS collected with a unique electrode in 5 mM (K₃[Fe(CN) s]^{-3/-4}). An equivalent circuit system (inset in figure S1) was used to fit the experimental EIS results. This circuit contains the ohmic resistance of electrolyte (R1), the Warburg impedance (Wd2), the charge-transfer resistance (R2), and the double layer capacitance (Q2). The fitting shows that the simulated line (dots) is in good agreement with the experimental impedance data (solid lines), as demonstrated in the selected circuit. In FIG. S1 is presented the bare gold electrode as it exhibits a low charge electron transfer resistance with the value of 4.7 kΩ cm⁻² (line a). After the immobilization of the probes, it is created a blocking step for affording a good stability of the probes and, as at the same time, for increasing the resistance of the double layer. [4] After this step, the modified gold electrode has been analyzed anew using EIS technique. However, the resistance in the surface of electrode according to the immobilization of P1 and P2 DNA probes on the gold electrode, have a new value R2. As it is presented in FIG. S1 was increased to 8.8 kΩ cm⁻² (line b), which is possible a result of the electrostatic repulsion between the negatively charged DNA and the redox mediator. Furthermore, after the blocking procedure and the deposition of thiol in the surface of the electrode, the value of R2 (12.6 kΩ cm²) is increased also in this case. This is a result of the double layer effect that it has been created by thiol which hindering the electron transfer (line c). Furthermore, in every case the value of double layer capacitance Q2 is increased, as far as more molecules were deposited in the surface of the gold electrode, as it is presented in table 1. Finally, once the genome of HBV has been recognized by the probes at the electrode surface, it is created the supramolecular complex DNA target-probe formation. The addition of the [Ru(phen)₂dppz]²⁺ complex allows its intercalation into ds-DNA groves owing to π-π stalking nature of the molecule In fact, dppz complexes with Ru(II) and Os(II) are only faintly luminescent in water, but becomes highly luminescent when intercalating ds-DNA.

TABLE 1
Electrodes modified with different
values of resistance to transfer of charge
(R2) and double-layer capacity (Q2)

| | R2 | Q2 |
|---|---|---|
| Elettrodi | [kQ cm⁻²] | [μF cm⁻²] |
| Au | 4.7 | 82.7 |
| Au_P1P2 | 8.8 | 140.3 |
| Au_P1P2_tiol | 12.6 | 166.1 |

### Analytical Performance

Further test was carried out with real sample of synthetic ds-genome of HBV. This sample bearing a starting concentration of 106 cps per reaction, and it was diluted to obtain testing solution concentrations of a final DNA amount of 0-1-5-10-100-1000-10000 cps per reaction, respectively. To assess the sensitivity of the proposed method, the ECL intensity is detected as a function of the HBV concentration. Electrochemical experiments were performed in a 3-electrodes cell (see experimental part for details), using a standard concentration 1000 cps per reaction. In this analytical performance the use of the K₂S₂O₈ was crucial such as is an important factor for the creation of radicals. In this case, we obtain a positive response of our system with an ECL intensity 4.93±0.32 a.u. (FIG. 2). The reduction of peroxydisulfate (eq 1) and [Ru(phen)₂dppz]²⁺ (eq 2) may take place, thus making the sequence of processes outlined by eqs 3-5 possible. Peroxydisulfate is going to be reduced and generate sulfate anion radical, either directly at the electrode (eq 1) or by mediation of [Ru(phen)₂dppz]²⁺ (eq 4). In line with the below hypothesis, an intense ECL signal was observed only when the negative potential was swept in the first scan obtaining the highest value of ECL intensity in every different concentration of synthetic genome of HBV. The equations 1-5 summarize the possible pathway for the production [Ru(phen)₂dppz]²⁺* when K₂S₂O₈ is used as the co-reactant in negative potential −0.80V vs Ag/AgCl reference electrode. The ECL intensity of the [Ru(phen)₂dppz]²/S₂O₈²⁻ system was found to be a function of S₂O₈²⁻ concentration (50 mM) and for 14 μM of [Ru(phen)₂dppz]²⁺, thus using this combination we achieved to qualify the synthetic genome of HBV. This result is ascribed to the fact that the persulfate ion is an effective co-reactant of [Ru(phen)₂dppz]²⁺ as well as an excellent quencher of the excited [Ru(phen)₂dppz]²⁺*.

S₂O₈²⁻+e⁻->SO₄·⁻+SO4²⁻ (1)

[Ru(phen)₂dppz]²⁺+e⁻->[Ru(phen)₂dppz]⁺ (2)

[Ru(phen)₂dppz]⁺+SO₄²⁻->[Ru(phen)₂dppz]²⁺+SO₄·⁻+SO₄²⁻ (3)

[Ru(phen)₂dppz]⁺+SO₄·⁻->[Ru(phen)₂dppz]²⁺·+SO₄²⁻ (4)

[Ru(phen)₂dppz]²⁺·->[Ru(phen)₂dppz]²⁺+hv (5)

For the further investigation of secondary factors that could affect the system of the invention, the pH effect was investigated, using two different pHs for the hybridization of the supramolecular complex formation. The pH can affect the enhancement of ECL signal in every kind of co-reagent. Also in this case, in which four different values of pH was used, the ECL signal for the same concentration of HBV synthetic genome (100 cps per reaction) was changed (FIG. S3). At pH<7.4, the presence of more H⁺ can modify the chemical balance in our system such as the creation of SO₄·⁻ species cannot be so effectively. On the other hand, for pH>7.4 the recognition of HBV synthetic genome by the specific P1 and P2 probes cannot be held in the surface of gold electrode so this effect brings the dramatically reduction of ECL signal value. Finally, for all ECL experiments has been used pH 7.4 such as it is observed the higher value of ECL intensity. As it is observed in FIG. 2, we repeat this experimental process, and we measure the electrochemical response also using higher/lower concentrations than the 1000 cps per reaction. In every case we observe different ECL intensity which is proportional of the ds-DNA concentration. The ECL intensity, which is detected by the PMT, is came from the reduction reaction between the K₂S₂O₈ (co-reagent) and the [Ru(phen)₂dppz]²⁺ (luminophores). In the equations 1-5 is described the possible pathway to produce light emission. The range of concentration which was held on the CV-ECL technique was 0-1000 cps per reaction. The maximum ECL value which is presented in FIG. 2 (dark blue line) for 1000 cps per reaction is instead of the minimum one (5 cps per reaction) which presented in FIG. 2 (red line) 0.41±0.08 a.u. In every case of these results, the experiment has been repeated more than 3 times in which is tested the repeatability of every measurement.

### Selectivity

In order to evaluate the selectivity of the proposed system, the biosensor was tested with an unspecific target, the Mycobacterium tuberculosis (MTB) synthetic clone at three different concentrations (10-100-1000 cps/reaction). The results prove that our chemical strategy is unique only for the supramolecular complex formation between probes and HBV synthetic genome. Thus, in the case of MTB there is no ECL signal response (FIG. S2 a-c). On the other hand, is fully observed the ECL signal response in the case of ds-HBV genome, such as it captured by two parallel and anti-parallel complementary P1 and P2 probes. In the last case, the [Ru(phen)₂dppz]²⁺ can be intercalated and quantify the presence of HBV in the modified surface of our gold electrode (FIG. S2d).

### Optimization of Experimental Variables

The analytical ECL response of the proposed system was evaluated. To assess the sensitivity of our method, the ECL intensity has been examined as a function of the logarithmic ds-HBV concentrations.

For the evaluation of the sensing capability, the limit of detection (LoD) was measured under the above-mentioned optimum conditions. The calibration curve was established by plotting the change in ECL intensity and logarithmic ds-HBV concentration. A good linear range between 5 cps per reaction and 10000 cps per reaction with a correlation coefficient of 0.987 is obtained. The LoD (S/N=3.3) has been calculated using the calibration plot (FIG. 3) in which the value is 2.7 cps per reaction, something that confirms the sensitivity of our method, as 37 times more efficient and sensitive than the PCR method.

### Applicability in Biological Fluids

Fetal Bovine Serum (FBS) were used for the applicability of the modified gold electrode for the determination of HBV. The same experimental procedure as described in experimental section has been followed for this analysis, with the main difference that the triplex formation has been done in FBS solution instead of PBS buffer. However, three different synthetic genomes of HBV concentrations (100-1000-10000 cps per reaction) have been examined using ECL technique. All our measurements in FBS have a great ECL response and follow our theory about the quantification of ds-HBV genome in PBS solution (FIG. S4). The results show satisfactory recoveries of HBV with the real standard deviation (RSD) in the range of 82.7-104.1% (table 2), thus confirming the efficient applicability of the proposed electrochemical DNA sensor in biological fluids.

TABLE 2
Different HBV concentrations in PBS
and FBS with Real Standard Deviation

| HBV | |
|---|---|
| concentration in | Real Standard |
| PBS/FBS | Deviation |
| [cps per reaction] | (RSD) |
| 100 | 82.7% |
| 1000 | 97.1% |
| 10000 | 104.1% |

### Real Samples Analysis

This experimental procedure has been held for the validation and quantification of HBV genome. The combination of P1 and P2 probes were properly designed to recognize specific genetic sequences on both the parallel and anti-parallel strands of the same target genome. The applicability and the validity of the proposed modified gold electrode was evaluated for the determination of extracted HBV sample. The real sample analysis was performed using CV technique and obtain the ECL response, where measurements were carried out using the set up and pretreatment protocol described.

The real samples control has been completed in the same range of concentrations, such as in FIG. 2. In the case of extracted HBV genome, the ECL intensity values for analytical samples are always lower than the synthetic one. The extracted sample does not include any vector having then the size of the HBV genome, corresponding to 3300 bps. However, the analytical sample HBV clone consists of the genome (3.3 kbps) cloned into a plasmid PBR322 vector (3.8 kbps) corresponding to a final circular structure of 7144 bps. This difference corresponds the highest intercalation of [Ru(phen)₂dppz]²⁺ in the grooves of synthetic HBV genome. The ECL intensity is 11 times lower in the case of extracted clone of HBV and this happens owing to the lower redox signal. According to the FIG. 4a, it can be verified the presence of the ds-DNA of HBV because of the quite high ECL intensity that is owed in different concentrations (0-1000 cps/ml). The electrochemical reaction (reduction) of our system takes place in same negative potential (−0.80 V) on which is observed also in FIG. 2. The ECL curves is the result of the reduction of K₂S₂O₈ and [Ru(phen)₂dppz]²⁺ as it is described on the equation (1-5). The extracted HBV genome has been studied and evaluated for the sensing capability and the limit of detection (LoD) was measured under the above-mentioned optimum conditions. The calibration curve was established by plotting between the change in ECL intensity and logarithmic ds-HBV concentration. A good linear range between 50 cps per reaction and 10000 cps per reaction with a correlation coefficient of 0.973 is obtained. The LoD (S/N=3.3) has been calculated using the calibration plot (FIG. 4b) in which the value is 60 cps per reaction, something that confirms the sensitivity of our method, as 1.7 times more efficient and sensitive instead of the PCR method. Generally, the ECL response of these two different genomes of HBV has been achieved with the comparison between two different LoDs. In the case of the synthetic HBV genome, we obtained a LoD around 2.7 cps/ml as it is described above. On the other hand, the LoD for the determination of extracted HBV genome, using the same pathway, is around 60 cps/ml. This huge difference between these two LoD values shows the different amount of the intercalated [Ru(phen)₂dppz]²⁺ in the grooves of HBV ds-DNA. The length of the HBV DNA has an important interruption in the ECL intensity.

The above examples are provided as an indication of the invention.

1-17. (canceled)

18. A method of detecting an analyte in a sample, wherein the analyte is a target whole genome of an organism, comprising:
a) contacting the sample containing the whole genome with at least two single-strand nucleic acid probes wherein each of the at least two single-strand nucleic acid probes is complementary to a corresponding portion of the target whole genome;
b) adding an electrochemiluminescence (ECL) active luminophore; and
c) determining a luminescent signal generated by the ECL active luminophore;
wherein the target whole genome is selected from a DNA or RNA genome.

19. The method of claim 1, wherein the target whole genome is single stranded (ss-) or double stranded (ds-).

20. The method of claim 1, wherein the single-strand nucleic acid probes are DNA, RNA, or PNA.

21. The method of claim 1, wherein the single-strand nucleic acid probes are immobilized by a surface linker.

22. The method of claim 21, wherein the surface linker is HS—(CH₂)n- wherein n is 6-18.

23. The method of claim 1, wherein the ECL active luminophore is a Ru(II), Ir(III), Re(I), or Os(II) coordination complex.

24. The method of claim 1, wherein:
when the target whole genome is a double-stranded genome, at least one of the at least two single-strand nucleic acid probes is complementary to a portion of the parallel strand of the double-stranded target whole genome and at least one of the at least two single-strand nucleic acid probes is complementary to a portion of the anti-parallel strand of the double-stranded target whole genome.

25. The method of claim 1, wherein the target whole genome is a viral or bacterial nucleic acid or parasite, eukaryotic cell whole genome.

26. The method of claim 25, wherein the viral nucleic acid is of Hepatitis B Virus (HBV).

27. The method of claim 26, wherein when the target whole genome is of HBV, the at least two single-strand nucleic acid probes are: at least one sequence is the sequence SEQ ID NO: 1 (GGTGAGTGATTGGAGGTT) and at least one sequence is SEQ ID NO: 2 (CACATCAGGATTCCTAGG).

28. The method of claim 27, wherein a linker is used with the sequence SEQ ID NO: 1 (GGTGAGTGATTGGAGGTT) and SEQ ID NO: 2 (CACATCAGGATTCCTAGG).

29. The method of claim 28, wherein the linker is HS—(CH₂)₆—.

30. The method of claim 1, wherein the ECL active luminophore has a ligand selected from dppz, 1,10-phenanthroline, and quinoxalino[2,3-f][1,10]phenanthroline.

31. The method of claim 1, wherein the ECL active luminophore is Ru(bpy)₂dppz]²⁺ or [Ru(phen)₂dppz]²⁺, wherein phen=1,10-phenanthroline, bpy=2,2′-bipyridine, and dppz=dipyrido[3,2-a:2′,3′-c]phenazine.

32. The method of claim 1, wherein the luminescence is electrochemically generated with sacrificial co-reactants.

33. The method of claim 32, wherein the co-reactant is K₂S₂O₈.

34. The method of claim 33, wherein the co-reactant is selected from a tertiary amine, tri-n-propylamine, 2-(dibutylamino)ethanol oxalate, and hydrogen peroxide.


SEQUENCE LISTING (2 sequences)

SEQ ID NO: 1 - DNA, 18 bp, unidentified
  source 1..18: mol_type = other DNA
ggtgagtgattggaggtt

SEQ ID NO: 2 - DNA, 18 bp, unidentified
  source 1..18: mol_type = other DNA
cacatcaggattcctagg